(19) United States
(12) Patent Application Publication
(10) Pub. No.: US 2016/0375016 A1
(43) Pub. Date: Dec. 29, 2016

(54) METHODS FOR REDUCING ANXIETY AND IMPULSIVITY IN SUBJECTS INITIATING TREATMENT WITH SEROTONIN REUPTAKE INHIBITORS

(71) Applicant: THE JOHNS HOPKINS UNIVERSITY, Baltimore, MD (US)

(72) Inventors: Adam I. Kaplin, Baltimore, MD (US); Craig W. Hendrix, Ellicott City, MD (US); Kristen A. Rahn, Baltimore, MD (US)

(21) Appl. No.: 14/900,636

(22) PCT Filed: Jun. 24, 2014
(86) PCT No.: PCT/US14/43853

§ 371 Date: Dec. 22, 2015

Related U.S. Application Data
(60) Provisional application No. 61/838,584, filed on Jun. 24, 2013.

(51) Int. Cl. A61K 31/496; A61K 31/343; A61K 31/381; A61K 31/357; A61K 31/137; A61K 31/138; A61K 31/135

(52) U.S. Cl. CPC A61K 31/496; A61K 31/138; A61K 31/343; A61K 31/135; A61K 31/357; A61K 31/137; A61K 31/381

(57) ABSTRACT

The present invention provides compositions and uses thereof for reducing anxiety and/or impulsivity, including, for example, suicidality, in a subject undergoing treatment with a serotonin reuptake inhibitor (SRI) comprising administering to the subject an effective amount of a SRI and an effective amount of a 5-HT1A receptor partial agonist/antagonist. In addition, the present invention provides dosing regimens for various SSRIs which can also reduce anxiety and/or impulsivity, including, for example, suicidality in a subject undergoing treatment with an SSRI. Kits including daily dosing regimens of various SSRIs are also provided.

### REFERENCE TO RELATED APPLICATIONS

This application claims the benefit of U.S. Provisional Patent Application No. 61/838,584, filed on Jun. 24, 2013, which is hereby incorporated by reference for all purposes as if fully set forth herein.

### BACKGROUND OF THE INVENTION

Clinical depression, including major depressive disorder (MDD), occurs in up to 8% of all children and adolescents and can negatively impact social, cognitive, and emotional development. Suicide, the worst potential outcome of severe depression, is the third leading cause of death of adolescents and early adults 10-24 years of age and the second leading cause for young adults ages 25-34. While cognitive behavioral therapy (CBT) is often sufficient to treat those with mild or moderate depression, in other cases treatment of MDD in children and adolescents often requires pharmacological intervention. The FDA has approved only one antidepressant for the treatment of MDD in children and adolescents 8-18 years old, the selective serotonin reuptake inhibitor (SSRI) fluoxetine (Prozac™). Another SSRI, escitalopram (Lexapro™) has FDA approval for the treatment of MDD in adolescents 12-17 years old.

Many psychotropic drugs, including SSRIs, prevent the reuptake of serotonin (5-HT) by blocking presynaptic 5-HT transporters, thereby increasing the availability of synaptic 5-HT to stimulate the postsynaptic neuron and, through additional mechanisms still not completely understood, producing the desired antidepressant effect. There is, however, a delay in the clinical onset of antidepressant effects, believed to be the result of excess synaptic 5-HT that activates 5-HT1A autoreceptors on the presynaptic neuron to halt the release of 5-HT. This delay correlates with the observed clinical delay in patients treated with SSRIs. The 2-4 week delay of treatment efficacy in SSRIs provides substantial patient risks, as studies have demonstrated a robust increase in suicidal ideation and impulsive attempts at self-harm (collectively termed “suicidality”) in patients under 25 during the first month of treatment with an SSRI, with a gradual decline in suicidality as exposure time increases.

In October 2004, the FDA enforced introduced a black box warning on SSRIs for children and adolescents due to studies showing increased suicidality in children and adolescents taking SSRIs (4%, versus 2% in placebo controls from a meta-analysis of 24 randomized double-blind clinical trials). Stressing the extremely rapid onset of emergent thoughts or behaviors of self-harm, the FDA issued guidelines that required weekly examinations to monitor suicidality during the first month of treatment. Erring on the side of safety, this FDA warning was extended to include young adults (18-24 years) in 2007. While these data underscore the potentially risky early side effects of antidepressants, a host of epidemiological data suggests SSRIs remain extremely useful in the treatment of depressed adolescents and children. A study comparing medical records over 10 years demonstrated that for every 1% increase in SSRI use in a population of 10-19 year olds from 1990 to 2000, there was a decrease of 0.23 completed suicides per 100,000 subjects. A similar study conducted in the United States and the Netherlands reported an inverse relationship between the changing number of prescriptions issued to adolescents for the treatment of MDD and the rates of suicide, specifically that a 22% reduction of prescriptions in both countries resulted in a 14% (2003-2004) and 49% (2003-2005) increase in suicide rates, respectively.

In addition, the 5-HTR1A gene is located on the long arm of chromosome 5 (5q11.2-13). A functional C(-1019)G variant has been reported. This polymorphism (rs6295) is a common SNP in the promoter region of the gene, within a 26 bp palindromic region, which binds the nuclear DEAF-1-related (NUDR) proteins and Hess5. The G allele abolishes repression by NUDR to produce higher expression of 5-HTR1A, which in turn enhances the negative feedback inhibition exerted by 5-HTR1A autoreceptors on serotonergic raphe neurons, thus leading to a decrease in serotonergic neurotransmission. This polymorphism can result in upregulation of the receptor leading to more significant blockade of the firing of serotonergic neurons in response to SRI's. Thus, there exists a subpopulation of subjects which may be at heighted risk for suicidality when initiating treatment with SRIs.

There still exists, therefore, a need for both close monitoring of children and adolescents initiating antidepressant therapy with SRIs, and the development of improved treatment strategies to minimize negative side effects and accelerate the onset of antidepressant effects.

### SUMMARY OF THE INVENTION

In accordance with an embodiment, the present invention provides a method for reducing anxiety and/or impulsivity in a subject initiating treatment with a serotonin reuptake inhibitor (SRI) comprising administering to the subject an effective amount of a SRI and an effective amount of a 5-HT1A receptor partial agonist/antagonist.

In accordance with another embodiment, the present invention provides a method for reducing suicidality in a subject initiating treatment with a serotonin reuptake inhibitor (SRI) comprising administering to the subject an effective amount of a SRI and an effective amount of a 5-HT1A receptor partial agonist/antagonist.

In accordance with an embodiment, the present invention provides method for reducing anxiety and/or impulsivity in a subject initiating treatment with a serotonin reuptake inhibitor (SRI), wherein the subject has been identified as having the C(-1019)G variant of the 5-HT1A receptor, comprising administering to the subject an effective amount of a SRI and an effective amount of a 5-HT1A receptor partial agonist/antagonist.

In accordance with another embodiment, the present invention provides method for reducing suicidality in a subject initiating treatment with a serotonin reuptake inhibitor (SRI), wherein the subject has been identified as having the C(-1019)G variant of the 5-HT1A receptor, comprising administering to the subject an effective amount of a SRI and an effective amount of a 5-HT1A receptor partial agonist/antagonist.

In accordance with a further embodiment, the present invention provides a method for reducing anxiety and/or impulsivity in a subject initiating treatment with a selective serotonin reuptake inhibitor (SSRI) other than fluoxetine comprising administering to the subject an effective amount of a SSRI in a dosing regimen which will provide a trough concentration of the SSRI in about 25 to 30 days which is therapeutically equivalent to administering 40 mg fluoxetine to a 70 kg subject once daily.

In accordance with still another embodiment, the present invention provides a method for reducing anxiety and/or impulsivity in a subject initiating treatment with a selective serotonin reuptake inhibitor (SSRI) other than fluoxetine, wherein the subject has been identified as having the C(-1019)G variant of the 5-HT1A receptor, comprising administering to the subject an effective amount of a SSRI in a dosing regimen which will provide a trough concentration of the SSRI in about 25 to 30 days which is therapeutically equivalent to administering 40 mg fluoxetine to a 70 kg subject once daily.

In accordance with still another embodiment, the present invention provides a method for reducing suicidality in a subject initiating treatment with a selective serotonin reuptake inhibitor (SSRI) other than fluoxetine, wherein the subject has been identified as having the C(-1019)G variant of the 5-HT1A receptor, comprising administering to the subject an effective amount of a SSRI in a dosing regimen which will provide a trough concentration of the SSRI in about 25 to 30 days which is therapeutically equivalent to administering 40 mg fluoxetine to a 70 kg subject once daily.

In accordance with a further embodiment, the present invention provides a kit for use in the methods described above, comprising 25 to 30 specific daily dosages of an SSRI other than fluoxetine, wherein each dose is labeled for use on a specific day of the dosing regimen.

### BRIEF DESCRIPTION OF THE DRAWINGS

FIG. 1 shows that mice treated with Fluoxetine are more anxious in an elevated plus maze compared to Control mice, but treatment with the 5-HT1AR antagonist WAY has no effect (1A). Co-administration of WAY with Fluoxetine reverses the anxiogenic effects of Fluoxetine treatment alone, and increases anxiolytic effects compared to Control and WAY mice. No differences in total distance traveled (1B) and maximum speed (1C) in the maze were observed between groups, indicating that drug treatment did not affect activity and mobility, respectively. Significantly different from Control at * (P<0.05); Significantly different from Fluoxetine at +(P<0.05), +++(P<0.001); Significantly different from WAY at ̂ (P<0.05). n=8-10 mice per group. FIG. 1D illustrates that the WAY-100365 reverses anxiety when coadministered with the SSRI fluoxetine, but does not reverse anxiety when co-administered with the SNRI reboxetine. n=8 mice per group.

FIG. 2 depicts the significant relationship between the log-transformed t1/2 of SSRIs vs. suicide-related events for the given SSRI in adults (2A) and children and adolescents (2B). A thorough literature search uncovered t_{1/2} data of 6 SSRIs for adult analysis (fluoxetine, citalopram, venlafaxine, sertraline, paroxetine, and fluvoxamine) and 4 SSRIs for pediatric analysis (fluoxetine, citalopram, sertraline, and paroxetine). A significant positive correlation was observed between the two factors in both populations (P<0.05). There is also a significant relationship between the inverse of the time to 90% of maximum concentration (Cₘₐₓ) of that drug when it is administered on a constant dosing schedule and the rate of SREs (2C, P<0.05).

FIG. 3 shows simulated time course of blood concentrations for citalopram, sertraline, paroxetine, fluvoxamine and venlafaxine based on a constant dosing regimen (i.e. how the drugs are currently prescribed according to manufacturer's instructions) and a titrated dosing regimen to closely match the pharmacokinetics of fluoxetine. The concentration-time course for fluoxetine was generated according to the manufacturer's instructions (Prozac Full Prescribing Information, 2013; pi.lilly.com/us/prozac.pdf).

FIG. 4 is a table depicting dosing regimens for paroxetine, citalopram, sertraline, venlafaxine, and fluvoxamine. If paroxetine, citalopram, sertraline, venlafaxine, and fluvoxamine are given based on the following table, their daily mean blood concentrations (Cave) will be matched to that of fluoxetine at 40 mg every 24 hours. Dose is in mg. Cₐᵥₑ is in ng/mL. Patient weight is assumed to be 70 kg. Cave for Venlafaxine is the combination of venlafaxine and its active metabolite, 0-desmethylvenlafaxine (labeling.pfizer.com/showlabeling.aspx?id=100).

### DETAILED DESCRIPTION OF THE INVENTION

Treatment of clinical depression with an SRI, including SSRIs, leads to an elevated risk of suicidality in child and adolescent subjects during the first phase of drug administration. A meta-analysis of 372 double-blind randomized placebo controlled trials of SSRIs for the treatment of depression, other psychiatric disorders, and non-psychiatric disorders reported odds ratios of 1.62 and 2.30 for suicidality and suicidal behaviors, respectively, in participants 24 years and younger (8% of the total subjects). Subjects ages ranged from 15-99, and one study found a 2.6% decrease in suicidal ideation and a 4.6% decrease in preparatory actions for suicide with every year of age increase of the subject. While the frequency of suicidality decreases as antidepressant exposure time increases, the marked increase in the odds ratio of suicidality remains elevated throughout the first 3 months of SSRI treatment. Correspondingly, in adult populations the initiation of SSRI treatment is paradoxically often associated with worsening anxiety, despite the approval of this class of medications to treat a variety of anxiety disorders. Acute SSRI administration causes a well-documented reduction in serotonergic neuron firing, and numerous clinical studies report that hyposerotonergic states (i.e. as ascertained by low CSF levels of the 5-HT metabolite 5-HIAA) correlate with increased impulsive, violent behavior, including suicide attempts and murders in patients with MDD. It is likely that increased anxiety and erratic behaviors in adults caused by initiation of SSRI treatment parallels the enhanced suicidality seen in children and adolescents, and that these behaviors operate through the same physiological mechanisms.

Pharmacological antidepressant treatment can be evaluated preclinically in rodents. Mice with genetically decreased levels of 5-HT neurotransmission display depressive behaviors, heightened anxiety to conditioned stimuli, and decreased anxiety to novel objects (i.e. impulsivity). Similar to humans, mice exhibit anxious behaviors following acute antidepressant treatment that disappear following chronic exposure to an SSRI.

In one or more embodiments, the present inventors have demonstrated a method of decreasing and reversing acute SRI-mediated anxiogenic behavior in mice using a 5-HT1A antagonist in conjunction with SRI treatment. Without being held to any particular theory, it is believed that by blocking the reuptake of 5-HT from serotonergic synapses, the 5-HT1A receptor antagonist prevents the characteristic decrease in serotinergic neuron firing during the initial days to weeks of antidepressant dosing.

Therefore, in accordance with an embodiment, the present invention provides a method for reducing anxiety, suicidality, and/or impulsivity in a subject initiating treatment with a serotonin reuptake inhibitor (SRI) comprising administering to the subject an effective amount of a SRI and an effective amount of a 5-HT1A receptor partial agonist/antagonist.

As used herein, the term “anxiety” refers to an uncomfortable and unjustified sense of apprehension that may be diffuse and unfocused and is often accompanied by physiological symptoms.

As used herein, the term “anxiety disorder” refers to or connotes significant distress and dysfunction due to feelings of apprehension, guilt, fear, etc. Anxiety disorders include, but are not limited to panic disorders, posttraumatic stress disorder, obsessive-compulsive disorder and phobic disorders.

As used herein, the term “depression” refers to a morbid sadness, dejection, or melancholy.

In accordance with another embodiment, the present invention provides a use of a composition comprising an effective amount of a serotonin reuptake inhibitor (SRI) and an effective amount of a 5-HT1A receptor partial agonist/antagonist, in a pharmaceutically acceptable carrier for reducing anxiety and/or impulsivity in a subject undergoing treatment with a SRI.

In accordance with another embodiment, the present invention provides a method for reducing suicidal ideation and/or self-harm in a subject initiating treatment with a serotonin reuptake inhibitor (SRI) comprising administering to the subject an effective amount of a SRI and an effective amount of a 5-HT1A receptor partial agonist/antagonist.

As used herein, the terms “suicidality” or “suicidal ideation and/or self-harm” are equivalent, and either refers to the appearance of suicidal thoughts and/or behavior following treatment, such as treatment with SRIs. For example, suicidal thoughts and behavior include, but are not limited to, the following: feeling that life is empty and/or wondering if life is worth living; thinking of suicide or death several times a week for several minutes; thinking of suicide or death several times a day in some detail; making specific plans for suicide; and attempting or succeeding in taking one's life.

As used herein, a “subject” refers to an individual awaiting or under medical care and treatment, such as treatment for symptoms of clinical depression. In some embodiments, the term “subject” means a child or adolescent patient about to initiate treatment for clinical depression. While the inventive methods are designed for human patients (e.g., male and female human patients), such methods are applicable to any suitable individual, which includes, but is not limited to, a mammal, such as a mouse, rat, rabbit, hamster, guinea pig, cat, dog, goat, cow, horse, pig, and simian.

The term “SRI or serotonin re-uptake inhibitor” means any psychotropic drug that contains serotonin reuptake activity. The term includes antidepressants that possess activities in addition to serotonin reuptake that would be expected to increase suicidality and would be remedied by a 5-HT1A receptor partial agonist/antagonist. Examples of SRIs include SSRIs, as well as SNRIs (serotonin and norepinephrine reuptake inhibitors) like Effexor and Cymbalta, for example.

The term “selective serotonin re-uptake inhibitors” or “serotonin-specific reuptake inhibitors” (SSRIs) means a class of compounds typically used as antidepressants in the treatment of depression, anxiety disorders, and some personality disorders.

SRIs and SSRIs are believed to increase the extracellular level of the neurotransmitter serotonin by inhibiting its reuptake into the presynaptic cell, increasing the level of serotonin in the synaptic cleft available to bind to the postsynaptic receptor. They have varying degrees of selectivity for the other monoamine transporters, with pure SSRIs having only weak affinity for the noradrenaline and dopamine transporter. Examples of drugs in this class include, but are not limited to, citalopram, dapoxetine, escitalopram, fluoxetine, fluvoxamine, indalpine, paroxetine, seratraline, zimelidine.

The term “5-HT1A antagonist” means a compound or molecule which inhibits the action of serotonin at the 5-HT1A receptor. Examples of compounds or molecules which have at least some antagonistic effect, either through direct antagonism, or partial agonist/antagonist activity on 5-HT1A receptors include, but are not limited to, pindolol, WAY100635, tandospirone (3aR,4S,7R,7aS)-rel-hexahydro-2-[4-[4-(2-pyrimidinyl)-1-piperazinyl]butyl]-4,7-methano-1H-isoindole-1,3(2H)-dione hydrochloride), and NAN-190 (1-(2-Methoxyphenyl)-4-(4-phthalimidobutyl)piperazine), p-MPPF, p-MPPI, (−)-NPPCC, WAY100135, WAY100635, DWAY, FCWAY, (S)-(+)-LY426965, (+/−)-LY426965, (R)-(−)-LY426965, cyanopindolol, alprenolol, risperidone, lecozotan (4-cyano-N-{2R-[4-(2,3-dihydrobenzo[1,4]-dioxin-5-yl)-piperazin-1-yl]-propyl}-N-pyridin-2-yl-benzamide HCl), buspirone, robalzotan (NAD-299), SB-649,915, and dotarizine.

In accordance with another embodiment, the present invention provides a use of a composition comprising an effective amount of a serotonin reuptake inhibitor (SRI) and an effective amount of a 5-HT1A receptor partial agonist/antagonist, in a pharmaceutically acceptable carrier for reducing suicidal ideation and/or self-harm in a subject undergoing treatment with a SRI.

As described above, a certain subpopulation of subjects may have a functional C(-1019)G variant of the 5-HTR1A receptor gene. The G allele abolishes repression by NUDR to produce higher expression of 5-HTR1A, which in turn enhances the negative feedback inhibition exerted by 5-HTR1A autoreceptors on serotonergic raphe neurons, thus leading to a decrease in serotonergic neurotransmission. This polymorphism can result in upregulation of the receptor leading to more significant blockade of the firing of serotonergic neurons in response to SRI's. Thus, there exists a subpopulation of subjects which may be at heighted risk for suicidality when initiating treatment with SRIs.

Thus, one of ordinary skill in the art would understand that this subpopulation of subjects would be identified through genetic testing. A sample of genetic material will be obtained from the subject and the sample would be assayed for the 5-HTR1A receptor gene. Using any known method, such as, for example, the polymorphism can be detected by allele specific hybridization, allele specific oligonucleotide ligation, primer extension, minisequencing, mass spectroscopy, heteroduplex analysis, single strand conformational polymorphism (SSCP), denaturing gradient gel electrophoresis (DGGE), oligonucleotide microarray analysis, temperature gradient gel electrophoresis (TGGE), and combinations thereof. If the sample shows that the subject has the C(-1019)G variant, the subject is identified as having a heightened risk of anxiety and/or impulsivity, or suicidality upon initiation of treatment for clinical depression with an SRI.

In accordance with an embodiment, the present invention provides method for reducing anxiety and/or impulsivity in a subject initiating treatment with a serotonin reuptake inhibitor (SRI), wherein the subject has been identified as having the C(-1019)G variant of the 5-HT1A receptor, comprising administering to the subject an effective amount of a SRI and an effective amount of a 5-HT1A receptor partial agonist/antagonist.

In accordance with another embodiment, the present invention provides method for reducing suicidality in a subject initiating treatment with a serotonin reuptake inhibitor (SRI), wherein the subject has been identified as having the C(-1019)G variant of the 5-HT1A receptor, comprising administering to the subject an effective amount of a SRI and an effective amount of a 5-HT1A receptor partial agonist/antagonist.

In accordance with one or more embodiments, the method of treatment of the subject beginning or undergoing SRI therapy, will be the administration of an effective amount of the SRI in combination with administration of an effective amount of the 5-HT1A receptor partial agonist/antagonist together or simultaneously. The SRI and 5-HT1A receptor partial agonist/antagonist can be in any pharmaceutically acceptable dosage forms.

In some embodiments, the SRI and 5-HT1A receptor partial agonist/antagonist can be in included together in one dosage form. In other embodiments, the subject can be administered a dose of either the SRI or 5-HT1A receptor partial agonist/antagonist first, followed by administration of a dose of the other a short period of time later.

In accordance with another embodiment, the 5-HT1A receptor partial agonist/antagonist can be administered prior to the initiation of SRI treatment for about 1 to about 5 days.

In accordance with one or more embodiments, the method of treatment of the subject beginning or undergoing SRI therapy, will be the administration of an effective amount of the SSRI in combination with administration of an effective amount of the 5-HT1A receptor partial agonist/antagonist, wherein the SRI and the 5-HT1A receptor partial agonist/antagonist are administered for a period of time of at least two weeks to about 30 days.

In an embodiment, the present invention provides a pharmaceutical composition comprising a SRI, or a salt, solvate, or stereoisomer thereof, and a 5-HT1A receptor partial agonist/antagonist compounds, or a salt, solvate, or stereoisomer thereof, and a pharmaceutically acceptable carrier.

In accordance with another embodiment, the present invention provides a use of a composition comprising an effective amount of a selective serotonin reuptake inhibitor (SSRI) other than fluoxetine in a pharmaceutically acceptable carrier for reducing anxiety and/or impulsivity, or suicidality in a subject undergoing treatment with a SSRI other than fluoxetine wherein an effective amount of a SSRI is given to the subject in a dosing regimen which will provide a trough concentration of the SSRI in about 25 to 30 days, and which is therapeutically equivalent to administering 40 mg fluoxetine to a 70 kg subject once daily.

Included within the compounds of the present invention are the tautomeric forms of the disclosed compounds, isomeric forms including diastereoisomers, and the pharmaceutically-acceptable salts thereof. The term “pharmaceutically acceptable salts” embraces salts commonly used to form alkali metal salts and to form addition salts of free acids or free bases. Examples of acids which may be employed to form pharmaceutically acceptable acid addition salts include such inorganic acids as hydrochloric acid, sulphuric acid and phosphoric acid, and such organic acids as maleic acid, succinic acid and citric acid. Other pharmaceutically acceptable salts include salts with alkali metals or alkaline earth metals, such as sodium, potassium, calcium and magnesium, or with organic bases, such as dicyclohexylamine. Suitable pharmaceutically acceptable salts of the compounds of the present invention include, for example, acid addition salts which may, for example, be formed by mixing a solution of the compound according to the invention with a solution of a pharmaceutically acceptable acid, such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. All of these salts may be prepared by conventional means by reacting, for example, the appropriate acid or base with the corresponding compounds of the present invention.

Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicines, the salts of the compounds of the present invention should be pharmaceutically acceptable salts. Other salts may, however, be useful in the preparation of the compounds according to the invention or of their pharmaceutically acceptable salts.

In addition, embodiments of the invention include hydrates of the compounds of the present invention. The term “hydrate” includes but is not limited to hemihydrate, monohydrate, dihydrate, trihydrate and the like. Hydrates of the compounds of the present invention may be prepared by contacting the compounds with water under suitable conditions to produce the hydrate of choice.

With respect to the pharmaceutical compositions described herein, the carrier can be any of those conventionally used, and is limited only by physico-chemical considerations, such as solubility and lack of reactivity with the active compound(s), and by the route of administration. The carriers described herein, for example, vehicles, adjuvants, excipients, and diluents, are well-known to those skilled in the art and are readily available to the public. It is preferred that the carrier be one which is chemically inert to the active agent(s), and one which has little or no detrimental side effects or toxicity under the conditions of use. Examples of the carriers include solid compositions such as solid-state carriers or latex beads.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

The choice of carrier will be determined, in part, by the particular pharmaceutical composition, as well as by the particular method used to administer the composition. Accordingly, there are a variety of suitable formulations of the pharmaceutical composition of the invention.

The dose of the compositions of the present invention also will be determined by the existence, nature and extent of any adverse side effects that might accompany the administration of a particular composition. Typically, an attending physician will decide the dosage of the pharmaceutical composition with which to treat each individual subject, taking into consideration a variety of factors, such as age, body weight, general health, diet, sex, compound to be administered, route of administration, and the severity of the condition being treated. By way of example, and not intending to limit the invention, the dose of the pharmaceutical compositions of the present invention can be about 0.001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight. In some embodiments, the dose of SRI administered to the subject ranges from about 5 mg/kg to 50 mg/kg. In some embodiments, the dose of 5-HT1A partial agonist/antagonist administered to the subject ranges from about 0.05 mg/kg to about 20 mg/kg, preferably about 0.1 to about 5 mg/kg.

The terms “treat,” and “prevent” as well as words stemming therefrom, as used herein, do not necessarily imply 100% or complete treatment or prevention of anxiety or impulsive behavior, or suicidal ideation and/or self-harm. Rather, there are varying degrees of treatment or prevention of which one of ordinary skill in the art recognizes as having a potential benefit or therapeutic effect. In this respect, the inventive methods can provide any amount of any level of treatment or prevention anxiety or impulsive behavior, or suicidal ideation and/or self-harm in a subject. Also, for purposes herein, “prevention” can encompass delaying the onset of the disease, or a symptom or condition thereof.

As used herein, the terms “effective amount” or “sufficient amount” are equivalent phrases which refer to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease, such as anxiety or impulsive behavior, or suicidality.

Buffers, acids and bases may be incorporated in the compositions to adjust pH. Agents to increase the diffusion distance of agents released from the composition may also be included.

The charge, lipophilicity or hydrophilicity of a composition may be modified by employing an additive. For example, surfactants may be used to enhance miscibility of poorly miscible liquids. Examples of suitable surfactants include dextran, polysorbates and sodium lauryl sulfate. In general, surfactants are used in low concentrations, generally less than about 5%.

The specific method used to formulate the novel formulations described herein is not critical to the present invention and can be selected from a physiological buffer (Feigner et al., U.S. Pat. No. 5,589,466 (1996)).

Therapeutic formulations of the product may be prepared for storage as lyophilized formulations or aqueous solutions by mixing the product having the desired degree of purity with optional pharmaceutically acceptable carriers, diluents, excipients or stabilizers typically employed in the art, i.e., buffering agents, stabilizing agents, preservatives, isotonifiers, non-ionic detergents, antioxidants and other miscellaneous additives, see Remington's Pharmaceutical Sciences, 16th ed., Osol, ed. (1980). Such additives are generally nontoxic to the recipients at the dosages and concentrations employed, hence, the excipients, diluents, carriers and so on are pharmaceutically acceptable.

The compositions can take the form of solutions, suspensions, emulsions, powders, sustained-release formulations, depots and the like. Examples of suitable carriers are described in “Remington's Pharmaceutical Sciences.” Id. Such compositions will contain an effective amount of the biopolymer of interest, preferably in purified form, together with a suitable amount of carrier so as to provide the form for proper administration to the patient. As known in the art, the formulation will be constructed to suit the mode of administration.

Buffering agents help to maintain the pH in the range which approximates physiological conditions. Buffers are preferably present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used.

Preservatives may be added to retard microbial growth, and may be added in amounts ranging from 0.2%-1% (w/v). Suitable preservatives for use with the present invention include phenol, benzyl alcohol, m-cresol, octadecyldimethylbenzyl ammonium chloride, benzyaconium halides (e.g., chloride, bromide and iodide), hexamethonium chloride, alkyl parabens, such as, methyl or propyl paraben, catechol, resorcinol, cyclohexanol and 3-pentanol.

Isotonicifiers are present to ensure physiological isotonicity of liquid compositions of the instant invention and include polhydric sugar alcohols, preferably trihydric or higher sugar alcohols, such as glycerin, erythritol, arabitol, xylitol, sorbitol and mannitol. Polyhydric alcohols can be present in an amount of between about 0.1% to about 25%, by weight, preferably 1% to 5% taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols; amino acids, such as arginine, lysine, glycine, glutamine, asparagine, histidine, alanine, ornithine, L-leucine, 2-phenylalanine, glutamic acid, threonine etc.; organic sugars or sugar alcohols, such as lactose, trehalose, stachyose, arabitol, erythritol, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thiosulfate; low molecular weight polypeptides (i.e., <10 residues); proteins, such as human serum albumin, bovine serum albumin, gelatin or immunoglobulins; hydrophilic polymers, such as polyvinylpyrrolidone, saccharides, monosaccharides, such as xylose, mannose, fructose or glucose; disaccharides, such as lactose, maltose and sucrose; trisaccharides, such as raffinose; polysaccharides, such as, dextran and so on.

Additional miscellaneous excipients include bulking agents, (e.g., starch), chelating agents (e.g., EDTA), antioxidants (e.g., ascorbic acid, methionine or vitamin E) and cosolvents.

Non-ionic surfactants or detergents (also known as “wetting agents”) may be added to help solubilize the therapeutic agent, as well as to protect the therapeutic protein against agitation-induced aggregation, which also permits the formulation to be exposed to shear surface stresses without causing denaturation of the protein. Suitable non-ionic surfactants include polysorbates (20, 80 etc.), polyoxamers (184, 188 etc.), Pluronic® polyols and polyoxyethylene sorbitan monoethers (TWEEN-20®, TWEEN-80® etc.). Non-ionic surfactants may be present in a range of about 0.05 mg/ml to about 1.0 mg/ml, preferably about 0.07 mg/ml to about 0.2 mg/ml.

Examples of diluents include a phosphate buffered saline, buffer for buffering against gastric acid in the bladder, such as citrate buffer (pH 7.4) containing sucrose, bicarbonate buffer (pH 7.4) alone, or bicarbonate buffer (pH 7.4) containing ascorbic acid, lactose, or aspartame. Examples of carriers include proteins, e.g., as found in skim milk, sugars, e.g., sucrose, or polyvinylpyrrolidone. Typically these carriers would be used at a concentration of about 0.1-90% (w/v) but preferably at a range of 1-10%

Generally, the ingredients are supplied either separately or mixed together in unit dosage form, for example, as a dry lyophilized powder or water-free concentrate in a sealed container, such as an ampule or sachet indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided, for example, in a kit, so that the ingredients may be mixed prior to administration.

An article of manufacture containing materials useful for the treatment of the disorders described above is provided. The article of manufacture comprises a container and a label. Suitable containers include, for example, bottles, vials, syringes and test tubes. The containers may be formed from a variety of materials such as glass or plastic. The container holds a composition which is effective for preventing or treating, for example, a wound or a joint disease and may have a sterile access port (for example, the container may be a vial having a stopper pierceable by a hypodermic injection needle). The label on or associated with the container indicates that the composition is used for treating the condition of choice. The article of manufacture may further comprise a second container comprising a pharmaceutically acceptable buffer, such as phosphate-buffered saline, Ringer's solution and dextrose solution. It may further include other materials desirable from a commercial and user standpoint, including buffers, diluents, filters, needles, syringes and package inserts with instructions for use.

At present, neither pindolol nor any other 5-HT1A receptor partial agonist/antagonist currently has FDA approval for use in children and adolescents, and pindolol has only been approved for use in the treatment of adult hypertension. In addition, in treatment, there exist vague and inconsistent practices employed by physicians when treating MDD in children and adolescents with antidepressants.

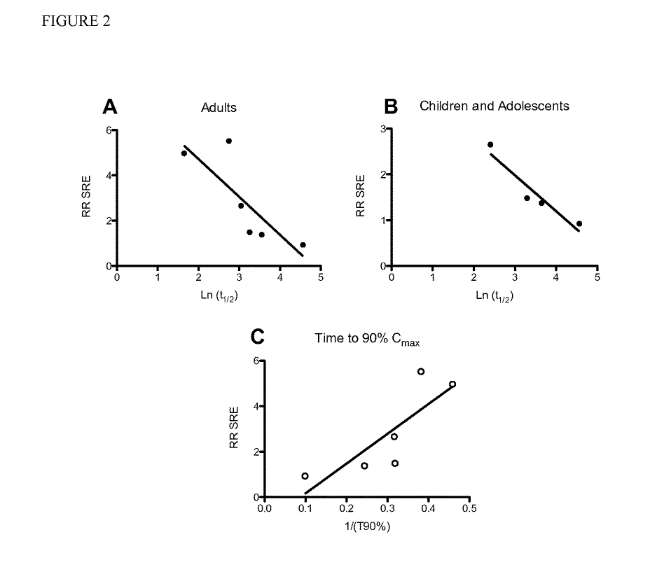

In accordance with one or more embodiments, the present invention provides a more thorough and researched approach to the treatment of childhood and adolescent clinical depression, including MDD, based on calculations conducted using computer modeling programs. When the present inventors plotted the blood clearance rate (1/_{t1/2}) of 5 SSRIs (fluoxetine, escitalopram, citalopram, sertraline, and paroxetine) vs. suicidality (also known as suicide related event or SRE) in pediatric populations prescribed SSRIs in double-blind placebo controlled studies (FIG. 2), a powerful and significant positive correlation between the two factors was found, indicating that the faster the clearance rate of an SSRI, and the shorter the half-life, the higher the chances of suicidality or SRE (R²=0.98, P=0.0014). The SSRI with the slowest clearance rate, longest half-life, and lowest incidence of suicidality was fluoxetine.

In accordance with an embodiment, the present invention provides a method for reducing anxiety and/or impulsivity in a subject initiating treatment with a selective serotonin reuptake inhibitor (SSRI) other than fluoxetine, comprising administering to the subject, an effective amount of a SSRI in a dosing regimen which will provide a trough concentration of the SSRI in about 25 to 30 days, which is therapeutically equivalent to administering 40 mg fluoxetine to a 70 kg subject once daily.

As used herein, the term “therapeutically equivalent to the fluoxetine trough concentration” means that a dosing curve was generated using the standard 40 mg/kg dose of fluoxetine on a 70 kg subject using a computer simulation program, such as MATLAB. After generation of a curve which achieved steady state in about 25 days, curves for other SSRIs were generated using the same parameters but using the pharmacokinetic data specific for the SSRI.

It will be understood by those of ordinary skill in the art that the dosing strategy of other SSRIs was determined as follows. The final dose at 30 days was targeted to be the companies recommended starting dose, then using a computer simulation program, the computer generated the best fit to the shape of the fluoxetine curve to reach that final dose. This provides a method of calculating the dosing for each SSRI of choice. Using the methods of the present invention, one can approximate the average dosing schedule for SSRIs with about a 24 hour half-life (e.g. Sertraline, Citalopram/Escitalopram) because they have similar pharmacokinetic profiles. The methods are less accurate with a SSRI having very short half-life (e.g. Venlafaxine).

For example, using the methods disclosed herein, the pharmacokinetic profile of fluoxetine was generated using MATLAB (Student Version, Release 14 with Service Pack 1; The MathWorks, Inc., Natick, Mass.) under the following assumptions: 1) the pharmacokinetics of fluoxetine can be described with a model of first-order absorption and Michaelis-Menten elimination: dC/dt=K01*(D/V)*exp (−K01*t)−Vm*C/(Km+C), where C is the concentration at time t after a single dose D; V, K01, Vm and Km are apparent volume of distribution, first-order absorption constant, maximum elimination constant, and Michaelis constant, respectively; 2) the body weigh was 70 kg and Prozac was administered as 40 mg once daily; 3) peak drug concentration (Cₘₐₓ), time to reach Cₘₐₓ (Tₘₐₓ) and half-life (t1/2) of fluoxetine following a single 40-mg dose of Prozac were 35 ng/mL, 7 hours, and 48 hours, respectively (Prozac Full Prescribing Information, 2013) and 4) the trough concentration at steady-state was in the range of 72-258 ng/mL (Prozac Full Prescribing Information, 2013). The final simulated model parameters were V, 1033 L; K01, 0.53/hr; Vm, 6.5 ng/mL-hr and Km, 448 ng/mL; the steady-state trough concentration of 133 ng/mL was apparently reached prior to Day 25.” It will be understood by those of ordinary skill that the calculations can be performed by any similar computer program in the art that permitted the input of equations (i.e. Micahelis Menton) and then could provide the necessary data when the values were plugged in.

In accordance with an embodiment, the dosing regimen will provide the trough concentration of the SSRI in about 25 to 30 days is therapeutically equivalent to the fluoxetine trough concentration of about 133 ng/ml.

It will be understood by those of ordinary skill in the art, that the SSRI used in the dosing regimen includes, but is not limited to, escitalopram, citalopram, sertraline, and paroxetine.

In accordance with an embodiment, the present invention provides a kit for use in the methods described above, comprising 25 to 30 specific daily dosages of an SSRI other than fluoxetine, wherein each dose is labeled for use on a specific day of the dosing regimen.

In accordance with an embodiment, the dosing regimen can comprise a regiment where the SRI and the 5-HT1A receptor partial agonist/antagonist are administered for a period of time of at least two weeks, followed by a first reduction in dosage of the 5-HT1A receptor partial agonist/antagonist, then after a second period of time ranging between two weeks and four weeks, a second reduction in dosage of the 5-HT1A receptor partial agonist/antagonist, followed by a third period of time wherein the in dosage of the 5-HT1A receptor partial agonist/antagonist is eliminated.

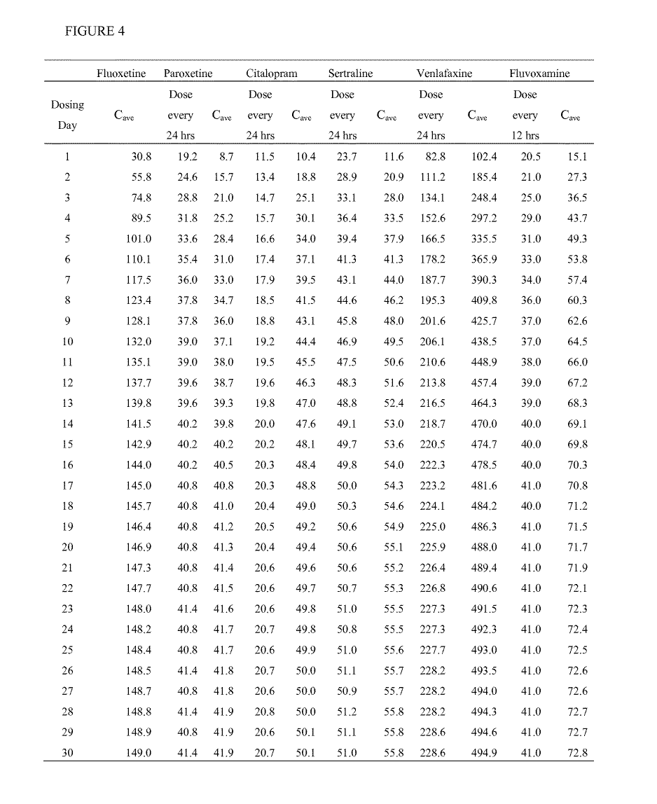

As shown in the table in FIG. 4, using the methods of the present invention, daily dosage regimens for any SSRI can be calculated using the data generated using the fluoxetine pharmacokinetic dosing curve. Thus, in one or more embodiments, the present invention provides a kit which includes specific daily doses for an SSRI other than fluoxetine, which are given on a specific day of the regimen, e.g., day 0, day 1, day 2, etc., and include a specific dose of the SSRI such that the pharmacokinetics of the SSRI approximate the pharmacokinetics of fluoxetine in the subject, and target drug levels of the SSRI reach steady state in about 25 to 30 days. For example, referring to FIG. 4, if a physician provided a kit of the present invention which was to deliver escitalopram, the kit would contain a dose for day 0 which provided 5.1 mg of escitalopram, a dose for day 1 which provided 6 mg of escitalopram, a dose for day 2 which provided 6.7 mg of escitalopram, and so on, for up to 30 days.

### EXAMPLES

Animals and Housing. 7-8 week old male C57BL/6 mice were obtained from the National Cancer Institute (Frederick, Md.) and housed in the Broadway Research Building Johns Hopkins animal facility. Housing and behavioral testing rooms were temperature controlled with a 12 h light/dark cycle, and all behavioral tests were conducted during the light cycle. Animal protocols were approved by the Johns Hopkins University School of Medicine Animal Care and Use Committee.

Drug administration. For 7 days prior to behavioral testing, vehicle (phosphate buffered saline, PBS) was delivered to each mouse via a 0.1 mL intraperitoneal injection. On testing days, vehicle (phosphate buffered saline), fluoxetine (20 ug/mL, Sigma-Aldrich), WAY-100635 (0.3 mg/kg, Sigma-Aldrich), and/or reboxetine (5 mg/kg, Sigma-Aldrich) were delivered 40 minutes prior to the commencement of testing.

Elevated Plus Maze. Mice (n=10) were tested on the elevated plus maze in an isolated behavior suite. Mice were placed in the center of the maze and given 5 minutes to freely explore the maze. Time spent in open and closed arms of the maze were tracked using ANY-maze software (Stoelting, Wood Dale, Ill.).

SSRI Clearance vs. Suicide Related Events. The odds ratio of suicide related events (SREs) in pediatric populations due to SSRI exposure was reported by Stone and colleagues in 2009 (BMJ, 2009. 339: p. b2880). The natural log-transformed t_{1/2} (ln(_{t1/2})) of 3 SSRIs in pediatric populations was calculated from the published pediatric t_{1/2} of citalopram (Guitierrez M, et al., “The pharmacokinetic profile of citalopram in adolescents and adults with major depressive disorder” in American Academy of Child and Adolescent Psychiatry, 2000 New York, N.Y.), sertraline (J Am Acad Child Adolesc Psychiatry, 2002. 41(9): p. 1037-44), paroxetine (J Am Acad Child Adolesc Psychiatry, 1999. 38(8): p. 952-9). Fluoxetine absorption, excretion, and distribution rates of in children and adolescents are equal to rates in adults (Expert Opin Drug Saf, 2004. 3(5): p. 495-504), so the half-life reported in adults was utilized in the present study. Blood clearance rates of 6 SSRIs in adults were calculated from the published adult t_{1/2} of fluoxetine, citalopram, sertraline, paroxetine, venlafaxine, and fluvoxamine (J Affect Disord, 2009. 114(1-3): p. 143-8). The natural log-transformed t_{1/2} of the SSRIs were plotted by the odds ratio of SREs associated with each individual SSRI for children and adults.

Antidepressant Dosing Simulation. The time course of the fluoxetine concentration in blood plasma was simulated based on the following assumptions: 1) the pharmacokinetics of fluoxetine can be described with a model of first-order absorption and Michaelis-Menten elimination: dC/dt=K01*(DN)*exp (−K01*t)−Vₘ*C/(Kₘ+C), where C is the concentration at time t after a single dose D; V, K01, Vm and Km are apparent volume of distribution, first-order absorption constant, maximum elimination constant, and Michaelis constant, respectively; 2) the body weigh was 70 kg and Prozac was administered as 40 mg once daily; and 3) peak drug concentration (Cₘₐₓ), time to reach Cₘₐₓ (Tₘₐₓ) and half-life (t_{1/2}) of fluoxetine following a single 40-mg dose of Prozac were 35 ng/ml, 7 hours, and 48 hours, respectively (Prozac Full Prescribing Information, 2013; pi.lilly.com/us/prozac.pdf). The final simulated model parameters were V, 1033 L; K01, 0.53/hr; Vₘ, 6.5 ng/mL-hr and Kₘ, 448 ng/mL; the simulated steady-state mean concentration of 149 ng/mL was apparently reached by Day 25.

The daily mean concentrations of fluoxetine as a percentage of the steady-state mean concentration were then set as the target of daily mean concentrations for dosing paroxetine, duloxetine, or citalopram. To simulate the dosing regimen, one-compartment pharmacokinetic model with first order absorption and elimination was used for sertraline [PMID:23436269], citalopram [PMID:6939299] and venlafaxine with its active metabolite, 0-desmethylvenlafaxine [http://labeling.pfizer.com/showlabeling.aspx?id=100]. V, t_{1/2} and Tₘₐₓ were assumed to be 1400 L, 26 hours, and 6.5 hours for sertraline [PMID:10674711], 840 L, 35 hours, and 4 hours for citalopram (Celexa package insert, 2012; frx.com/pi/celexa_pi.pdf), and 426 L [http://labeling.pfizer.com/showlabeling.aspx?id=100], 16.9 hours[PMID:23512639], and 8 hours [PMID:23512639] for venlafaxine, respectively. Since the metabolic pathway of paroxetine (Paxil package insert, 2013; http://us.gsk.com/products/assets/us_paxil.pdf) and fluvoxamine [PMID:10674711] at the therapeutic dose may be saturated, Michaelis-Menten kinetic model was used to simulate their dosing regimen. Cₘₐₓ, Tₘₐₓ, and t_{1/2} were assumed to be 17.6 ng/ml (Ref 37, i.e., PMID:2530793), 6.3 hours (Ref 37, i.e., PMID:2530793), and 16 hours (PMID:433623) for paroxetine after a single 30 mg dose; and 30 ng/ml, 6 hours, and 19 hours (PMID: 1485372) for fluvoxamine after a single 50 mg dose, respectively.

MATLAB Student Version Release 14 with Service Pack 1 (The MathWorks, Inc., Natick, Mass.) was used to find out Michaelis-Menten kinetic parameters and all dosing regimens. Phoenix WinNonlin 6.3 (Certara, L.P., St. Louis, Mo.) was used to generate the time-course of drug concentrations for a given dosing regimen.

Statistical Analyses. Statistical analyses were completed using GraphPad Prism 5.0 (La Jolla, Calif.). Comparisons between groups in the animal studies were made with one-way ANOVA, and P values <0.05 were considered statistically significant. The Johns Hopkins Institute for Clinical and Translational Research Biostatistics Center provided statistical support.

### Example 1

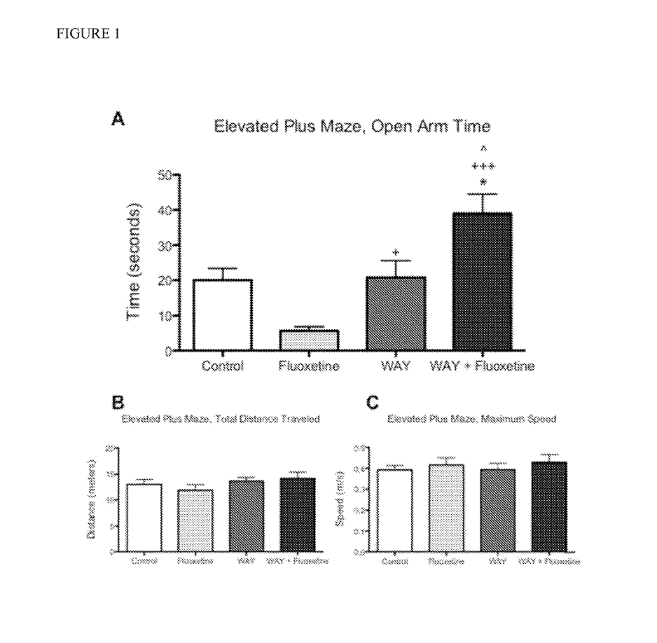

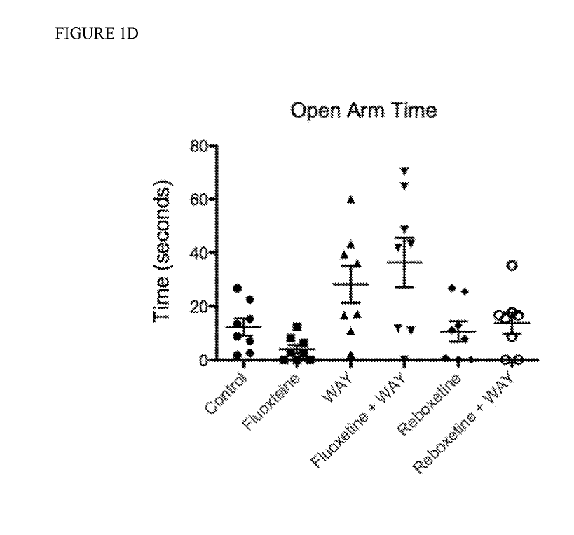

Chronic administration of SSRIs has an anxiolytic effect on mouse behavior, while acute administration of SSRIs has an anxiogenic effect on mouse behavior. The present studies confirmed that acute administration of fluoxetine is anxiogenic in 8 week-old mice as assessed by time spent in open arms of the elevated plus maze (EPM). Mice acutely treated with fluoxetine (20 mg/kg) spent 72% less time in the open arms compared to Control mice injected with saline only (5.67±3.68 vs. 20.03±10.12 seconds, respectively) (FIG. 1). Administration of the potent 5-HT1AR antagonist WAY-100635 (0.3 mg/kg) had no effect on time spent in the open arms compared to Control mice (20.82±15.18 vs. 20.03±10.12 seconds, respectively). Co-administration of WAY-100635 with fluoxetine, however, reversed the acute negative, anxiogenic effects of acute fluoxetine and WAY+fluoxetine treated mice spent significantly more time in the open arms of the maze compared to mice treated with fluoxetine alone (38.9±15.94 vs. 5.67±3.68 seconds, respectively, P<0.001). Total distance traveled in the elevated plus maze (open and closed arm distance combined) and maximum speed were not changed between groups (FIGS. 1B and C), indicating that the fluoxetine and WAY treatments affected anxiety but not activity and mobility.

Acute SSRI exposure decreases serotonergic output, whereas chronic administration and 5-HT1A autoreceptor downregulation SSRIs lead to increased levels of serotonin by blocking reuptake. To confirm that the anxiolytic effect of WAY-100635 was specific to SSRIs and not selective norepinephrine reuptake inhibitors (SNRIs), the acute effects of reboxetine and WAY+reboxetine treatments were assessed. While co-administration of WAY-100635 and fluoxetine caused a significant increase in the amount of time the mice spent in the open arm compared to fluoxetine alone (P<0.01, 36.4±26.1 vs. 4.0±4.5 seconds, respectively), mice given WAY-100635 with reboxetine spent equal time in the open arms of the maze as compared to mice given reboxetine alone or saline (13.79±11.3 vs. 10.6±10.8 seconds vs 12.3±3.2, respectively) (FIG. 1D).

### Example 2

SSRI-mediated SREs are well documented in pediatric populations [21]. Additionally, the blood clearance rates (i.e. half-life or t_{1/2}) of some SSRIs in pediatric populations have been reported. Here, we plotted the natural log of the t_{1/2} of 6 SSRIs (fluoxetine, citalopram, venlafaxine, sertraline, paroxetine, and fluvoxamine) vs. SREs in adult populations prescribed SSRIs (FIG. 2A). Limited data is available regarding the pharmacokinetics of SSRIs in pediatric populations, but of the 4 SSRIs for which data is published (fluoxetine, citalopram, sertraline, and paroxetine), we plotted the natural log of the t_{1/2} vs. SREs in pediatric populations prescribed SSRIs (FIG. 2B). We found a significant positive correlation between the two factors in both populations, indicating that the faster the clearance rate of a SSRI, the higher the chances of an SRE occurring (P<0.05). Furthermore, we found a significant relationship between the inverse of the time to 90% of maximum drug concentration (Cₘₐₓ) as a result of a constant dosing schedule and the rate of SREs for the given SSRI (P<0.05, FIG. 2C).

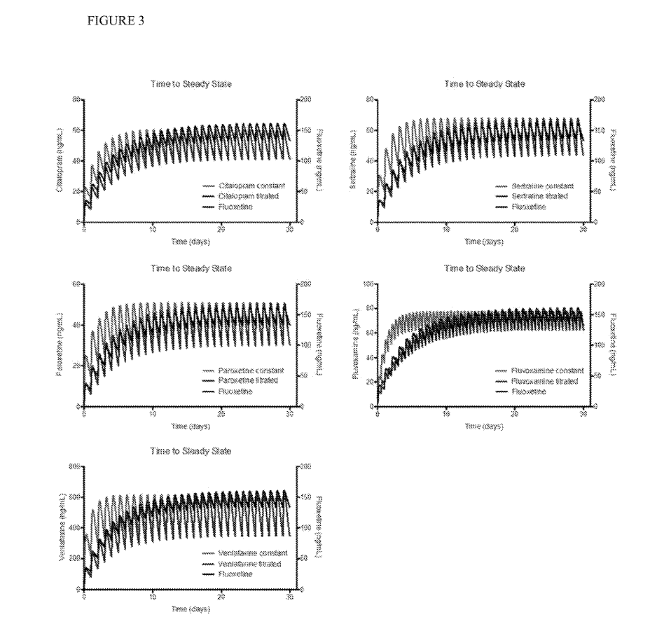

Fluoxetine, the antidepressant with the longest half-life, is the SSRI with the lowest relative risk of SREs (0.92) in pediatric populations. Since it is not possible to change the pharmacokinetic properties of the other SSRIs to approximate those of fluoxetine, we instead try to use a dose loading strategy to make other SSRIs approximate that of fluoxetine. We generated a dosing curve of fluoxetine following the standard and recommended dosing regimen of 40 mg per day using the computer program MATLAB. Steady state of drug concentration was reached after 25 days. Based on the generated fluoxetine curve, dosing regimens for paroxetine, duloxetine, and escitalopram were simulated to make the trough blood concentration of each of these drugs reach the steady state in a way similar to that of fluoxetine (FIG. 3). These simulated dosing regimens for paroxetine, duloxetine, and escitalopram have been converted into a table (FIG. 4) to serve as the dosing regimens of the present invention for the first month of SSRI treatment.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for reducing anxiety and/or impulsivity in a subject undergoing treatment with a serotonin reuptake inhibitor (SRI) comprising administering to the subject in need thereof, an effective amount of a SRI and an effective amount of a 5-HT1A receptor partial agonist/antagonist, and a pharmaceutically acceptable carrier.

2. (canceled)

3. The method of claim 1, wherein the SRI and the 5-HT1A receptor partial agonist/antagonist are administered together.

4. The method of claim 1, wherein the SRI and the 5-HT1A receptor partial agonist/antagonist are administered for a period of time of at least two weeks to about 30 days.

5. The method of claim 1, wherein the SRI and the 5-HT1A receptor partial agonist/antagonist are administered for a period of time of at least two weeks, followed by a first reduction in dosage of the 5-HT1A receptor partial agonist/antagonist, then after a second period of time ranging between two weeks and four weeks, a second reduction in dosage of the 5-HT1A receptor partial agonist/antagonist, followed by a third period of time wherein the in dosage of the 5-HT1A receptor partial agonist/antagonist is eliminated.

6. The method of claim 1, wherein the SRI is selected from the group consisting of fluoxetine, escitalopram, citalopram, sertraline, paroxetine, and venlafaxine.

7. The method of claim 1, wherein the 5-HT1A receptor antagonist is selected from the group consisting of: pindolol, WAY100635, tandospirone (3aR,4S,7R,7aS)-rel-hexahydro-2-[4-[4-(2-pyrimidinyl)-1-piperazinyl]butyl]-4,7-methano-1H-isoindole-1,3(2H)-dione hydrochloride), and NAN-190 (1-(2-Methoxyphenyl)-4-(4-phthalimidobutyl)piperazine), p-MPPF, p-MPPI, (−)-NPPCC, WAY100135, WAY100635, DWAY, FCWAY, (S)-(+)-LY426965, (+/−)-LY426965, (R)-(−)-LY426965, cyanopindolol, alprenolol, risperidone, lecozotan (4-cyano-N-{2R-[4-(2,3-dihydrobenzo[1,4]-dioxin-5-yl)-piperazin-1-yl]-propyl}-N-pyridin-2-yl-benzamide HCl), buspirone, robalzotan (NAD-299), SB-649,915, and dotarizine.

8. The method of claim 5, wherein the SRI is selected from the group consisting of fluoxetine, escitalopram, citalopram, sertraline, paroxetine, and venlafaxine.

9. The method of claim 5, wherein the 5-HT1A receptor antagonist is selected from the group consisting of: pindolol, WAY100635, tandospirone (3aR,4S,7R,7aS)-rel-hexahydro-2-[4-[4-(2-pyrimidinyl)-1-piperazinyl]butyl]-4,7-methano-1H-isoindole-1,3(2H)-dione hydrochloride), and NAN-190 (1-(2-Methoxyphenyl)-4-(4-phthalimidobutyl)piperazine), p-MPPF, p-MPPI, (−)-NPPCC, WAY100135, WAY100635, DWAY, FCWAY, (S)-(+)-LY426965, (+/−)-LY426965, (R)-(−)-LY426965, cyanopindolol, alprenolol, risperidone, lecozotan (4-cyano-N-{2R-[4-(2,3-dihydrobenzo[1,4]-dioxin-5-yl)-piperazin-1-yl]-propyl}-N-pyridin-2-yl-benzamide HCl), buspirone, robalzotan (NAD-299), SB-649,915, and dotarizine.

10. A method for reducing anxiety and/or impulsivity, or suicidality in a subject undergoing treatment with a SSRI other than fluoxetine, the method comprising administering to a subject undergoing treatment with a SSRI other than fluoxetine in need thereof, an effective amount of a selective serotonin reuptake inhibitor (SSRI) other than fluoxetine in a pharmaceutically acceptable carrier wherein an effective amount of the SSRI is given to the subject in a dosing regimen which will provide a trough concentration of the SSRI in about 25 to 30 days, and which is therapeutically equivalent to administering 40 mg fluoxetine to a 70 kg subject once daily.

11. The method of claim 10, wherein the dosing regimen will provide a trough concentration of the SSRI in about 25 to 30 days equivalent to the fluoxetine trough concentration in the range of 72 ng/ml to about 258 ng/ml.

12. The method of claim 10, wherein the trough concentration of the SSRI in about 25 to 30 days equivalent to the fluoxetine trough concentration is about 133 ng/ml.

13. The method of claim 10, wherein the SSRI is selected from the group consisting of escitalopram, citalopram, sertraline, and paroxetine.

14.-15. (canceled)

16. A method for reducing suicidal ideation and/or self-harm in a subject undergoing treatment with a SRI in need thereof, comprising administering to the subject an effective amount of a serotonin reuptake inhibitor (SRI) and an effective amount of a 5-HT1A receptor partial agonist/antagonist, in a pharmaceutically acceptable

17. The method of claim 16, wherein the SRI and the 5-HT1A receptor partial agonist/antagonist are administered together.

18. The method of claim 16, wherein the SRI and the 5-HT1A receptor partial agonist/antagonist are administered for a period of time of at least two weeks to about 30 days.

19. The method of claim 16, wherein the SRI and the 5-HT1A receptor partial agonist/antagonist are administered for a period of time of at least two weeks, followed by a first reduction in dosage of the 5-HT1A receptor partial agonist/antagonist, then after a second period of time ranging between two weeks and four weeks, a second reduction in dosage of the 5-HT1A receptor partial agonist/antagonist, followed by a third period of time wherein the in dosage of the 5-HT1A receptor partial agonist/antagonist is eliminated.

20. The method of claim 16, wherein the SRI is selected from the group consisting of fluoxetine, escitalopram, citalopram, sertraline, paroxetine, and venlafaxine.

21. The method of claim 16, wherein the 5-HT1A receptor antagonist is selected from the group consisting of: pindolol, WAY100635, tandospirone (3aR,4S,7R,7aS)-rel-hexahydro-2-[4-[4-(2-pyrimidinyl)-1-piperazinyl]butyl]-4,7-methano-1H-isoindole-1,3(2H)-dione hydrochloride), and NAN-190 (1-(2-Methoxyphenyl)-4-(4-phthalimidobutyl)piperazine), p-MPPF, p-MPPI, (−)-NPPCC, WAY100135, WAY100635, DWAY, FCWAY, (S)-(+)-LY426965, (+/−)-LY426965, (R)-(−)-LY426965, cyanopindolol, alprenolol, risperidone, lecozotan (4-cyano-N-{2R-[4-(2,3-dihydrobenzo[1,4]-dioxin-5-yl)-piperazin-1-yl]-propyl}-N-pyridin-2-yl-benzamide HCl), buspirone, robalzotan (NAD-299), SB-649,915, and dotarizine.

22. The method of claim 19, wherein the SRI is selected from the group consisting of fluoxetine, escitalopram, citalopram, sertraline, paroxetine, and venlafaxine.

23. The method of claim 19, wherein the 5-HT1A receptor antagonist is selected from the group consisting of: pindolol, WAY100635, tandospirone (3aR,4S,7R,7aS)-rel-hexahydro-2-[4-[4-(2-pyrimidinyl)-1-piperazinyl]butyl]-4,7-methano-1H-isoindole-1,3(2H)-dione hydrochloride), and NAN-190 (1-(2-Methoxyphenyl)-4-(4-phthalimidobutyl)piperazine), p-MPPF, p-MPPI, (−)-NPPCC, WAY100135, WAY100635, DWAY, FCWAY, (S)-(+)-LY426965, (+/−)-LY426965, (R)-(−)-LY426965, cyanopindolol, alprenolol, risperidone, lecozotan (4-cyano-N-{2R-[4-(2,3-dihydrobenzo[1,4]-dioxin-5-yl)-piperazin-1-yl]-propyl}-N-pyridin-2-yl-benzamide HCl), buspirone, robalzotan (NAD-299), SB-649,915, and dotarizine.
